(19) United States
(12) Patent Application Publication
(10) Pub. No.: US 2023/0053966 A1
(43) Pub. Date: Feb. 23, 2023

(54) INGESTIBLE MEDICAL DEVICE

(71) Applicant: Celero Systems, Inc., Lincoln, MA (US)

(72) Inventors: Benjamin D. Pless, Lincoln, MA (US); Daniel Bacher, Walpole, MA (US)

(21) Appl. No.: 17/795,420

(22) PCT Filed: Jan. 27, 2021
(86) PCT No.: PCT/US2021/015224

§ 371 Date: Jul. 26, 2022

Related U.S. Application Data
(60) Provisional application No. 62/966,120, filed on Jan. 27, 2020.
(60) Provisional application No. 62/966,146, filed on Jan. 27, 2020.

(51) Int. Cl. A61M 31/00; A61N 1/32; A61N 1/05

(52) U.S. Cl. CPC A61M 31/002; A61N 1/325; A61N 1/0509; A61M 2205/50; A61M 2205/3523; A61M 2205/3303

(57) ABSTRACT

An ingestible medical device that includes an ingestible capsule. Within the ingestible capsule is a non-refillable drug dispenser containing a therapeutic agent. The capsule can also include a real-time clock. A controller within the capsule can be operatively connected to the drug dispenser and can include a memory module and a processor operable to execute a software application stored on the memory module to control the operation of the drug dispenser to administer the therapeutic agent according to a real-time schedule measured via the real-time clock. The ingestible medical device can also include an iontophoretic circuit or system for drug delivery as well as physiological sensors to detect the occurrence or worsening of a medical condition.

### CROSS-REFERENCE TO RELATED APPLICATIONS

The present application claims priority to U.S. Provisional Application No. 62/966,120 filed on Jan. 27, 2020 and U.S. Provisional Application No. 62/966,146 filed on Jan. 27, 2020. All the above-referenced applications are incorporated herein by reference in their entirety.

### TECHNICAL FIELD

The present application relates to an ingestible capsule for drug delivery in the intestine either on a timed basis.

### BACKGROUND

There are various clinical situations in which delivery of a drug on timed basis can improve drug efficacy, reduce negative side effects, and improve clinical outcomes. Traditional oral medications can have compliance issues, particularly if the medications have to be taken multiple times during the day or the medical condition is generally asymptomatic.

### SUMMARY

In certain aspects, the present disclosure provides an ingestible medical device that automatically delivers medication at selected times to improve therapeutic value and compliance. Such an aspect provides controlled drug delivery where the drug can be released in a precise and controlled manner

### BRIEF DESCRIPTION OF DRAWINGS

FIG. 1 is a block diagram of an exemplary ingestible medical device according to an aspect of the present disclosure.

FIG. 2A is a perspective view of an exemplary ingestible medical device according to an aspect of the present disclosure and FIG. 2B is an inverted view of FIG. 2A.

FIG. 3 is a block diagram of an exemplary ingestible medical device according to an aspect of the present disclosure.

FIG. 4 is a block diagram of an exemplary ingestible medical device according to an aspect of the present disclosure where the device has attached to the intestinal wall.

FIG. 5 is a block diagram of an exemplary ingestible medical device according to an aspect of the present disclosure where the device has attached to the intestinal wall.

### DETAILED DESCRIPTION

As used herein with respect to a described element, the terms “a,” “an,” and “the” include at least one or more of the described elements including combinations thereof unless otherwise indicated. Further, the terms “or” and “and” refer to “and/or” and combinations thereof unless otherwise indicated. In addition, when an element is referred to as being “attached to,” “mounted on,” “disposed on,” “connected to,” or in “communication with” another element, it can be directly attached to, mounted on, disposed on, connected to, or in communication with the other element or intervening elements may also be present unless otherwise indicated. By “operatively connected” is meant two or more components are electrically, mechanically, or electromechanically connected to each other. By “substantially” is meant that the shape or configuration of the described element need not have the mathematically exact described shape or configuration of the described element but can have a shape or configuration that is recognizable by one skilled in the art as generally or approximately having the described shape or configuration of the described element. As such “substantially” refers to the complete or nearly complete extent of a characteristic, property, state, or structure. The exact allowable degree of deviation from the characteristic, property, state, or structure will be so as to have the same overall result as if the absolute characteristic, property, state, or structure were obtained. A “patient” as described herein includes a mammal, such as a human being. An “an ingestible medical device” and “ingestible capsule” as used herein is a device and capsule respectively that is not just capable of being ingested but rather is suitable for swallowing and entering into the gastrointestinal tract as known by one of skill in the art.

There are various clinical situations in which delivery of drug either on a timed basis has the potential to improve drug efficacy, reduce negative side effect, and improve clinical outcomes. One ideal location for controlled drug delivery is in the intestine, where the drug can be directed absorbed by the intestinal tissue either through active or passive mechanisms.

In an aspect, an ingestible device is provided that contains an electrical circuit folded into a small capsule suitable for swallowing. Upon being ingested, the capsule can pass through the patient's stomach and dissolve in the intestine thereby releasing the electronic circuit and other components. Once released, the electronic circuit can attach to the mucosal layer of the intestine where it can reside for a temporary period of time, such as up to one week, for example. The capsule can also include a non-refillable drug dispenser (also referred to herein as a “drug dispenser” or “drug reservoir”) containing at least one therapeutic agent. In certain aspects, the electrical circuit can contain a controller and a clock to control delivery of the therapeutic agent from the drug dispenser for time-scheduled delivery. The schedule for delivery of the therapeutic agent may be pre-programmed into the electrical circuit before ingestion or may be wirelessly communicated to the circuit while inside the patient's GI tract.

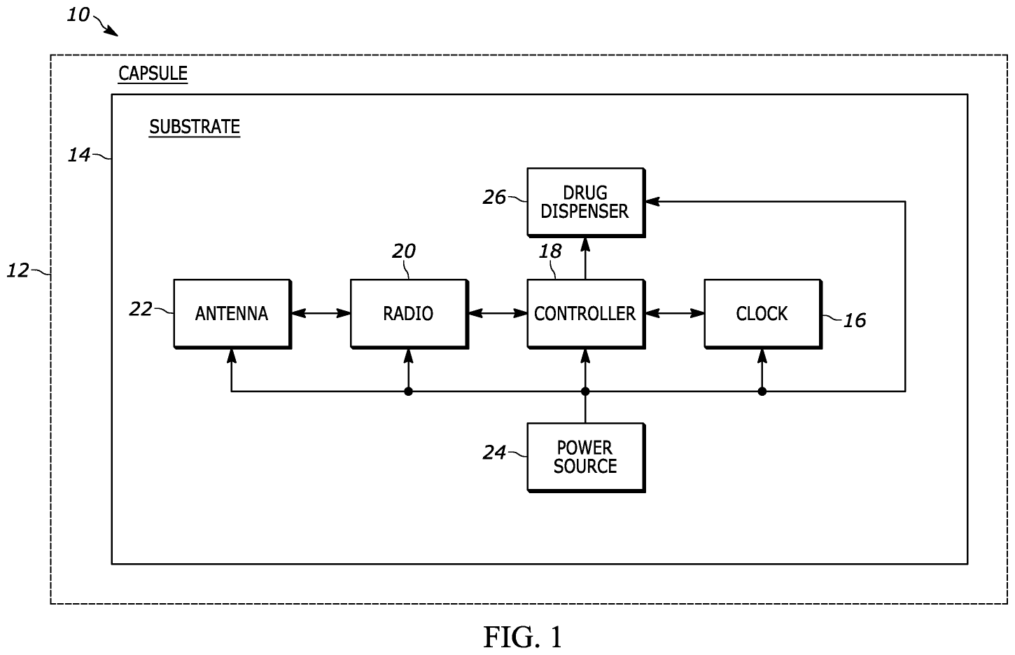

In particular, an ingestible medical device can comprise a non-refillable drug dispenser containing a therapeutic agent, a real-time clock, and a controller operable to execute a software application to control the operation of the drug dispenser to administer the therapeutic agent according to a real-time schedule via the real-time clock. In particular and with reference to FIG. 1, an ingestible medical device 10 can comprise an ingestible capsule 12 containing a substrate 14. Device 10 can also include a non-refillable drug dispenser 26 contained within capsule 12 that contains a therapeutic agent. The non-refillable drug dispenser is a reservoir that does not include any ports or similar structures that allow the drug dispenser to be re-filled from a location external to the patient's body, such as a syringe containing a therapeutic agent that is injected into the drug dispenser from outside the patient's body. Device 10 can also comprise controller 18 mounted on substrate 14 and operatively coupled to drug dispenser 26. Device 10 can further include a real-time clock 16 and a communication module comprising, for example, a radio 20 and an antenna 22. Ingestible medical device 10 can also include a power source 24 operatively connected to controller 18, clock 16, radio 20 and antenna 22. It should be noted that the antenna and other suitable components can have at least a portion mounted off-substrate or on-capsule.

The ingestible capsule can be a gelatin capsule or can have another type of suitable enteric coating that allows it to pass through the acidic environment of the stomach and then dissolve upon reaching the neutral pH environment of the intestine. Non-limiting examples of enteric coatings include cellulose acetate phthalate (CAP), poly(methacrylic acid-co-methyl methacrylate), cellulose acetate trimellitate (CAT), poly(vinyl acetate phthalate) (PVAP), or hydroxypropyl methylcellulose phthalate. The ingestible capsule can have various forms so long as the capsule is suitable for ingestion. For example, the capsule can have any suitable size for swallowing such as between size 000 to size 4. The ingestible capsule can also take the form of a tablet similar in configuration to tablets taken orally for ingestion of a drug. For example, the tablet can be approximately 10 mm long and approximately 7 mm thick.

The substrate can assume a folded compact shape in the capsule. As such, the substrate can be fabricated from a flexible biodegradable material such as, for example, silk, cellulose, or another biodegradable material. Conductors in the substrate can also be fabricated from a biodegradable material, such as, for example, magnesium alloy. Alternatively, the substrate and/or the conductors can be fabricated from a non-biodegradable material. Mechanical links can be integrally formed on the substrate, separating groups of electrical components. The mechanical links can be fabricated of a biodegradable material. Alternatively, the mechanical links can include a biogalvanic material, such that when energized, the mechanical links rapidly corrode. The substrate can be a flexible printed circuit board. The top side of the substrate can be coated with a thin hydrophobic coating such as a 5 micron thick layer of parylene. The bottom side of the substrate can be coated completely or in part (for example just at one end) with a mucoadhesive such as a hydrogel that contains or is largely formulated from a muco-adhesive such as poly(butadiene-maleic anhydride-co-L-DOPA) (PBMAD) or Carbopol, and other suitable mucoadhesives. When the substrate unfurls, the mucoadhesive layer can cause the bottom side of the substrate to lightly adhere to the mucosa on the wall of the intestine. Alternatively, anchors such as barbs, tines, etc. can be connected to the substrate to temporarily fixate the substrate to the intestinal wall. The top side of the substrate can be disposed towards the lumen of the intestine. Individual circuits (such as a battery; a memory module; a controller and a Bluetooth module) as a group can be encapsulated in a thin and conformal coat of a material like silicone or epoxy. This can make the substrate biocompatible and smooth so that chyme and other contents of the intestine can flow with minimal resistance over the top of the substrate. Most of the functionality of the ingestible medical device can be contained in a custom integrated circuit.

The substrate can also be a low-profile ribbon that is folded, rolled or otherwise packed into the capsule as described in U.S. patent application Ser. No. 17/007,122 (filed on Aug. 31, 2020) and incorporated by reference herein (see paragraphs [0018]; [0020]; [0021]; [0029] and [0035]). The ribbon can be fabricated from a flexible material such as silk, cellulose, or polyimide, for example. The material can be biodegradable or non-biodegradable. Although the ribbon itself can be low profile in the sense that it is thin and flat, it can have rigid sections and flexible sections therebetween. An optional anchor can be connected to the substrate and sized and configured to attach to the mucosal surface of the patient's intestine.

The drug dispenser can be any suitable drug dispenser including an iontophoretic device (described in more detail below), a MEMS drug delivery device, valve systems, osmotic plug pistons, electrolytical pumps, or combinations thereof. The ingestible capsule can contain a single drug dispenser with the same therapeutic agent or different therapeutic agents or can contain a plurality of drug dispensers, with each dispenser containing the same therapeutic agent, different concentrations or release characteristics (e.g. extended, fast) of the same therapeutic agent, or different therapeutic agents. The drug dispenser can be mounted on the substrate. Unless otherwise specified, the drug dispenser can be located on either top or bottom surface so long as the therapeutic agent can be released from the dispenser and absorbed into the intestines (e.g. delivered into the lumen or delivered into the intestinal wall). The drug dispenser can be loaded in the capsule as the final manufacturing step.

The ingestible medical device can also include a clock, such as a real-time clock. A real-time clock (RTC) is an electronic device, most often in the form of an integrated circuit, that measures the passage of time and is synchronized with actual time of day (such as universal time). The RTC can use a crystal oscillator, a resonant circuit, or a micromechanical resonator. The real-time clock can be used for a number of functions including time drug delivery and time stamping events and logs stored in the memory. The RTC can be mounted on the substrate.

Regarding the controller, the controller can comprise a memory module and a processor operable to execute a software application stored on the memory module to control the operation of the drug dispenser to administer the therapeutic agent according to a real-time schedule measured via the real-time clock as described in more detail below. The controller can be mounted on the substrate or can otherwise be contained within the ingestible capsule. The processor can be any type of suitable logic circuit including, for example, a microprocessor. The controller, the RTC and other components can be formed at least partially as an application specific integrated circuit (ASIC).

Regarding the communication module, such a module can include a non-specific radio module, or other wireless communication module that can communicate with an external control unit. For example, a Bluetooth radio can communicate directly with a smart phone or other Bluetooth enabled device outside the patient's body. The Bluetooth radio can be a 2.4 GHz Bluetooth or a BLE (Bluetooth Low Energy) radio. The communication link can allow a physician to configure the ingested device to collect certain data and to retrieve the data after it has been collected. In an alternative configuration, wireless data telemetry can be achieved using a sub-GHz frequency radio (specifically 400-900 MHz, including 433 MHz radios) communicating to a small receiver, such as a key fob-sized receiver that could be worn by the patient or otherwise placed on the patient's person that mediates data transfer between the ingested device and a smartphone via Bluetooth.

Regarding the power source, such a component can be an internal battery or an external power source. The power source can be operatively connected to the non-refillable drug dispenser, controller, and real-time clock to supply electrical power to each of these components.

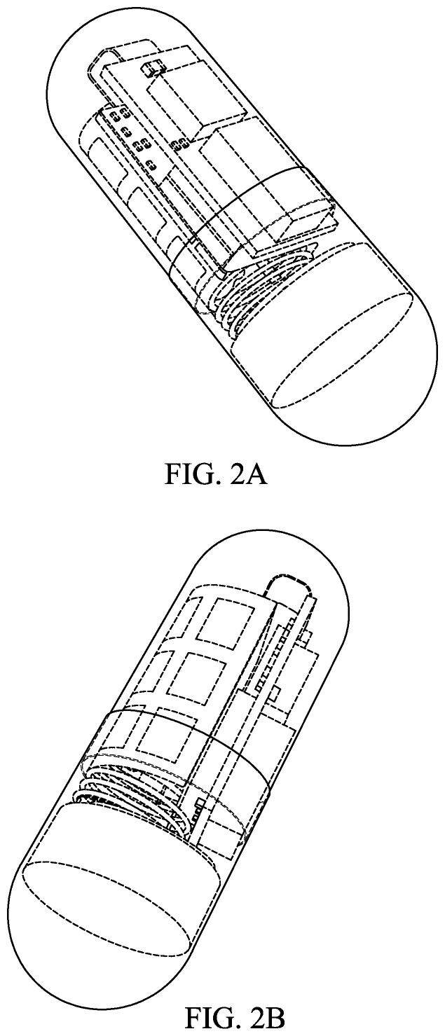

FIGS. 2A and 2B illustrate an exemplary arrangement of certain components of an ingestible medical device 21. For example, device 21 can comprise a capsule 23 within which is contained a controller 25, a power controller in the form of a battery 27, and a drug dispenser 29. Disposed about the outer surface of capsule 23 is an antenna 31. The device can comprise other components as described below.

An external device or external control unit can be used to wirelessly communicate with the ingestible medical device. For example, a non-specific radio module, or other wireless communication module of the ingestible medical device can be configured to communicate with an external device, such as a smartphone. The controller can be configured to control the operation of the radio to send information to the external device and the external device can be used to send instructions to the controller.

The ingestible capsule can also optionally include sensors to monitor on or more physiological parameters associated with the medical condition. Non-limiting examples of physiological parameters include electrical cardiac activity, heart rate, heart rate variability, respiratory monitoring (e.g. respiratory rate), saturated oxygen, intestinal tissue color, central temperature, bodily motions as detected from the intestine, or combinations thereof. Physiological parameters can be measured by an electrocardiogram (ECG) sensor, an accelerometer, a photoplethysograph (PPG) sensor, a temperature sensor, or combinations thereof. Upon detecting physiological parameters indicating onset or worsening of the medical condition, the ingested device can release a therapeutic agent via a drug dispenser as well as send out alerts to the patient and/or a caregiver.

The medical condition can be any suitable condition where use of an ingestible medical device is suitable and/or advantageous. In particular, the medical condition can be a condition where timed drug delivery is advantageous such as when compliance is an issue. For example, hypertension is a medical disorder where traditional oral drugs are helpful but compliance is an issue for many reasons. For example, hypertension generally does not have symptoms so patients may not be motivated to take their medications. In addition, the time of day that the medication is delivered is important. In fact, delivery of hypertension medication may be advantageous while the patient is asleep which presents clear difficulties.

The following is a non-limiting example of how an ingestible medical device that comprises an ingestible capsule can release a therapeutic agent according to a time schedule. The ingestible medical device can comprise a power source, such as a battery; a pre-loaded non-refillable drug dispenser; a real-time clock; a microprocessor with on-board memory; and a communication module, such as a radio and an antenna. The processor can communicate with the drug dispenser to electrically command release of medication from the drug dispenser. Components of the ingestible medical device can be disposed on a ribbon-like substrate, for example, that is folded up and held in the ingestible capsule. Prior to ingestion by the patient, through use of the radio, the real-time clock can be synchronized with the patient's local time and the medication release schedule can be downloaded into the microprocessor's memory. Upon ingestion by the patient, the capsule can travel through the stomach into the intestine where it can dissolve, releasing the substrate that unfurls and adheres to the intestinal wall. The processor can log the timing of the substrate's unfurling. By the use of polling, interrupts or other techniques, the processor can command the drug dispenser to release the medication on the time schedule stored in its memory. For example, in the case of the medical condition being hypertension, the following representative dosages of doxazosin may be delivered from the drug dispenser into the intestine at the respective times: 1 mg at 10:00 pm; 2 mg at 3:00 am; and 1 mg at 7 am. Such times and values are exemplary and other times and dosages may be delivered or adjusted to improve blood pressure in individual patients.

In certain aspects, the therapeutic agent is released via iontophoresis. Iontophoresis is a non-invasive technology for delivering therapeutic agent(s) using a small electric current, which causes an electrical field. In general, delivering such therapeutic agents through iontophoresis involves applying an electromotive force that transports ions through tissue. An iontophoresis circuit or system comprises a treatment electrode, a return electrode, and a power source. Activation of the power source causes the treatment and return electrodes to obtain opposite charge polarities. The opposite charge polarities cause the therapeutic agent to ionize. The ionized therapeutic agent is then driven into tissue, such as tissue of the stomach or intestine as a result of the repulsive force between the treatment and return electrodes.

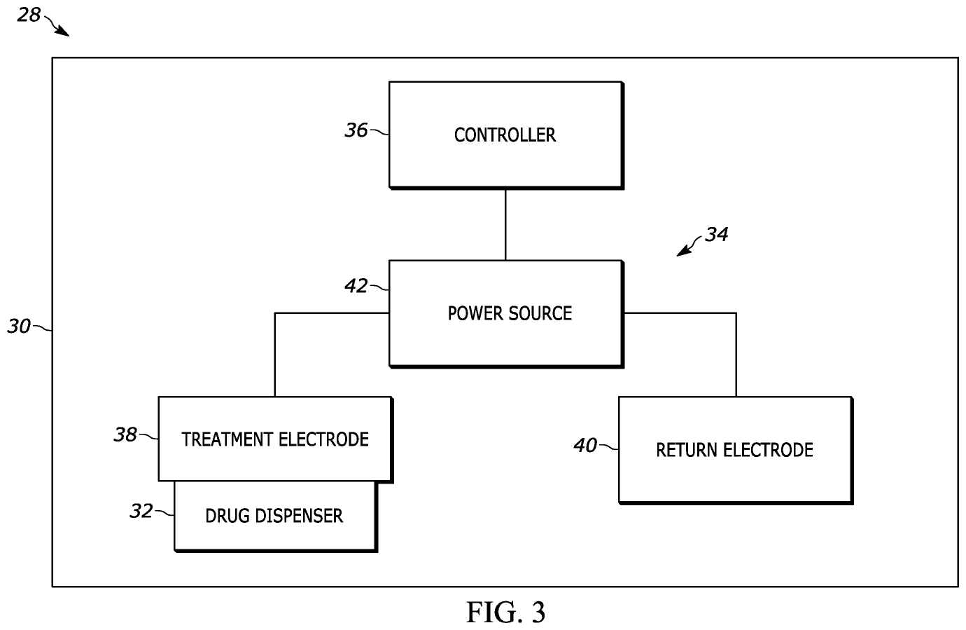

Referring to FIG. 3, an ingestible medical device 28 comprises an ingestible capsule 30 containing a non-refillable drug dispenser 32, an iontophoresis circuit 34, and a controller 36. The non-refillable drug dispenser can contain a polarizable therapeutic agent. Iontophoresis circuit 34 can comprise a treatment electrode 38 having a first polarity and operatively connected to the drug dispenser and a return electrode 40 having a second polarity opposite of the first polarity. The treatment electrode should be in sufficient contact with the therapeutic agent such that is can deliver an electric current to motivate the therapeutic agent into gastrointestinal tissue as illustrated in FIG. 3. Ionotophoresis circuit 34 can further include a power source 42 that is operatively connected to treatment electrode 38 and return electrode 40, such as between the treatment electrode and the return electrode, as illustrated in FIG. 3. The power source can include a battery and/or a low-voltage, DC or AC signal generator having positive and negative terminals that are in electrical communication with the respective treatment electrode and return electrode (or vice versa). Controller 36 is operatively connected to power source 42 of ionotophoresis circuit 34.

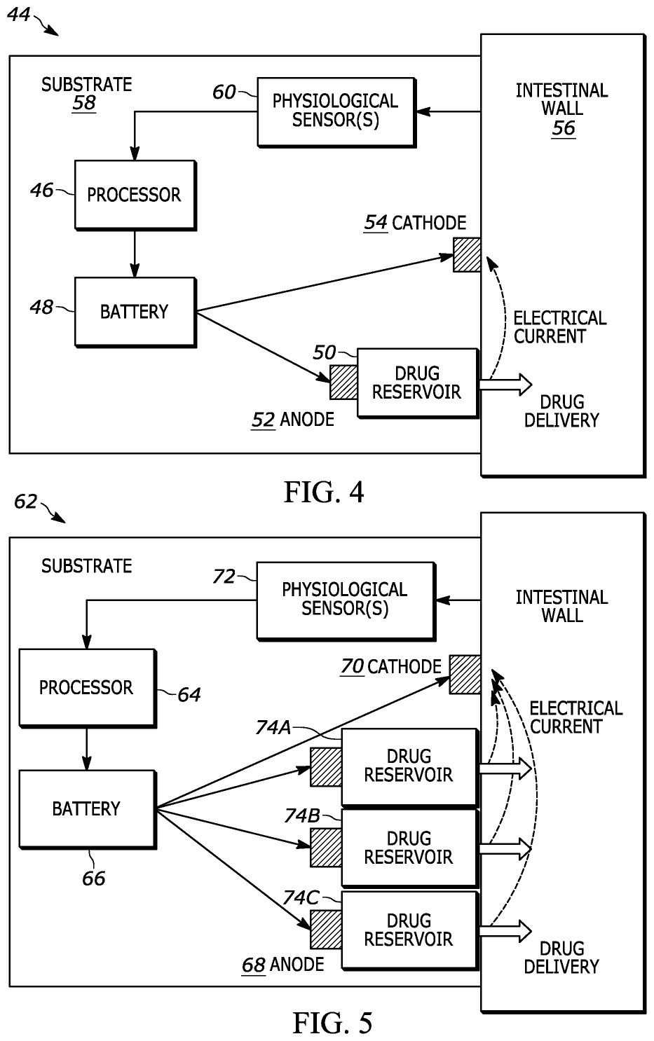

Referring to FIG. 4, in certain aspects. an ingestible medical device 44 comprises a controller including a processor 46, a drug delivery dispenser or reservoir 50, and an iontophoresis system or circuit comprising a battery 48, a treatment electrode (which is indicated as an anode) 52, a return electrode (which is indicated as a cathode) 54, which is in contact with the intestinal wall 56 when the capsule is ingested, reaches the intestine, and the substrate unfurls. The ingestible medical device 44 can also include at least one physiological sensor 60. It is understood that the treatment electrode could serve as cathode and the return electrode could serve as anode. Components of the ingestible medical device can be mounted on a flexible substrate 58. Once reaching the intestine and after the substrate unfurls, drug reservoir 50 can mechanically couple to intestinal wall tissue 56. The processor can interpret sensor signals from the physiological sensor and if a clinical event is detected, the processor can generate a control signal to responsively deliver a therapeutic agent. The processor can also generate a drug release control signal on a timed basis at scheduled intervals (the ingestible medical device can include a clock as described above). In response to a control signal from the processor, the battery connected to the drug reservoir can apply an electrical voltage via the anode across the reservoir membrane and through the intestinal wall tissue to facilitate drug absorption. The volume of drug delivered may be controlled through the voltage magnitude and duration of the voltage applied to the drug reservoir.

Referring to FIG. 5, in certain aspects, an ingestible medical device 62 comprises a controller including a processor 64, a battery 66, a treatment electrode (which is indicated as an anode) 68, a return electrode (which is indicated as a cathode) 70, and at least one physiological sensor 72. In this aspect, the ingestible medical device comprises a plurality of drug reservoirs 74. Each drug reservoir can be independently controlled by control signals from the processor. Voltage can be applied to two or more drug reservoirs simultaneously to increase the rate of drug delivery compared to a single drug reservoir. Voltage can also be applied to drug reservoirs sequentially to sustain long-term drug delivery, e.g. as one drug reservoir empties, the processor initiates drug delivery through another drug reservoir.

Numerous therapeutic agents can be delivered through an ingestible medical device for a variety of clinical applications. In one such application, a therapeutic agent can be responsively delivered iontophoretically in the intestine in response to a physiological sensor detecting a physiological parameters indicative of a specific medical condition. For example, an anticoagulant can be delivered in response to sensors detecting atrial fibrillation or other cardiac arrythmia. In an additional application, a hypertension drug can be delivered at optimal time points in the patient's circadian rhythm in a periodic fashion.

Although the above-described embodiments have been described in with respect to a device implanted in the intestine of a patient, the ingestible medical device may be configured to reside in the stomach. For example, the ingestible capsule can have anchors, such as fins or barbs, disposed thereon to increase the size of the capsule so that it is large enough to interfere with passage through the pylorus for the desired residency time.

Each of the disclosed aspects and embodiments of the present disclosure may be considered individually or in combination with other aspects, embodiments, and variations of the disclosure. Further, while certain features of embodiments and aspects of the present disclosure may be shown in only certain figures or otherwise described in the certain parts of the disclosure, such features can be incorporated into other embodiments and aspects shown in other figures or other parts of the disclosure. Along the same lines, certain features of embodiments and aspects of the present disclosure that are shown in certain figures or otherwise described in certain parts of the disclosure can be optional or deleted from such embodiments and aspects. Additionally, when describing a range, all points within that range are included in this disclosure. Further, unless otherwise specified, none of the steps of the methods of the present disclosure are confined to any particular order of performance. Furthermore, all references cited herein are incorporated by reference in their entirety.

1-14. (canceled)

15. An ingestible medical device to treat a patient with a medical condition comprising:
an ingestible capsule;
a non-refillable drug dispenser contained within the capsule and comprising a therapeutic agent;
a real-time clock; and
a controller operatively connected to the non-refillable drug dispenser and comprising a memory module and a processor operable to execute a software application stored on the memory module to control the operation of the non-refillable drug dispenser to administer the therapeutic agent according to a real-time schedule measured via the real-time clock.

16. The ingestible medical device of claim 15, further comprising a power source operatively connected to the non-refillable drug dispenser, controller, and real-time clock to supply electrical power to the non-refillable drug dispenser, controller, and real-time clock.

17. The ingestible medical device of claim 15, wherein the software application is configured to control the non-refillable drug dispenser to administer the therapeutic agent on a predetermined calendar/time schedule measured via the real-time clock.

18. The ingestible medical device of claim 15, wherein the software application is configured to control the non-refillable drug dispenser to administer the therapeutic agent at predetermined times of day measured via the real-time clock.

19. The ingestible medical device of claim 15, further comprising a radio and an antenna operatively connected to the controller and configured to communicate wirelessly with an external control unit.

20. The ingestible medical device recited in claim 19, wherein the external control unit is configured to program the controller through wireless communication and control the operation of the medical device while ingested through wireless communication.

21. The ingestible medical device recited in claim 15, further comprising a substrate comprising a flexible printed circuit board.

22. The ingestible medical device of claim 15, wherein the controller is programmed to timestamp data with time information from the real-time clock and store the timestamped data in the memory module.

23. The ingestible medical device of claim 15, further comprising an anchor disposed on the ingestible capsule sized and configured to interfere with passage of the ingestible capsule through the pylorus of the patient for a desired residency time.

24. The ingestible medical device of claim 15, wherein the ingestible capsule is configured to reside in the stomach of the patient for a temporary period of time.

25. An ingestible medical device comprising:
an ingestible capsule;
a non-refillable drug dispenser contained within the ingestible capsule and comprising a therapeutic agent;
an iontophoresis circuit comprising:
a treatment electrode having a first polarity and operably connected to the non-refillable drug dispenser;
a return electrode having a second polarity opposite of the first polarity;
a power source adapted to provide an electric current via the treatment electrode sufficient to motivate the therapeutic agent into gastrointestinal tissue; and
a controller operatively connected to the power source of the ionotophoresis circuit.

26. The ingestible medical device of claim 25, further comprising a real-time clock operatively connected to the controller.

27. The ingestible medical device of claim 25, wherein the controller comprises a memory module and a processor operable to execute a software application stored on the memory module to control the operation of the non-refillable drug dispenser to administer the therapeutic agent according to a real-time schedule measured via the real-time clock.

28. The ingestible medical device of claim 25, further comprising a sensor contained with the capsule and configured to detect a physiological parameter related to the medical condition.

29. The ingestible medical device of claim 28, wherein the controller is programmed to receive a sensor signal detected by the sensor of the physiological parameter to actuate or adjust release of the therapeutic agent from the non-refillable drug dispenser into a stomach of the patient based on the sensor signal.

30. The ingestible medical device of claim 25, wherein the non-refillable drug dispenser comprises a plurality of non-refillable drug dispensers.
